(19) United States
(12) Patent Application Publication
(10) Pub. No.: US 2021/0005289 A1
(43) Pub. Date: Jan. 7, 2021

(54) METHODS, DEVICES, AND SYSTEMS FOR MANAGING AND TRACKING OF COLLECTION, PROCESSING, AND MOVEMENT OF SAMPLES DURING CLINICAL TRIALS

(71) Applicant: TruLab, Inc., Durham, NC (US)

(72) Inventors: Richard Alan Graham, Burlingame, CA (US); Scott Anthony Ogle, Durham, NC (US); Ho Kwang Chung, Durham, NC (US)

(21) Appl. No.: 16/918,227

(22) Filed: Jul. 1, 2020

Related U.S. Application Data
(60) Provisional application No. 63/019,457, filed on May 4, 2020.
(60) Provisional application No. 62/870,559, filed on Jul. 3, 2019.

(51) Int. Cl. G16H 10/20; G16H 40/63; G06K 19/06; G16H 40/67

(52) U.S. Cl. CPC G16H 10/20; G16H 40/63; G16H 40/67; G06K 19/06037; G06K 19/06028

(57) ABSTRACT

Methods, devices, and systems for solving the problem of management and tracking of collection, processing, and movement of biological samples during clinical trials are disclosed herein. According to one embodiment, a method is implemented on one or more servers. The method includes receiving first biological sample data from a first mobile device. The first mobile device includes a camera for capturing at least a portion of the first biological sample data and a first graphical user interface (GUI) configured for displaying the first biological sample data. The method further includes storing the first biological sample data within a distributed ledger. The distributed ledger may include at least one block chain.

### CROSS-REFERENCE TO RELATED APPLICATION

This application claims the benefit of U.S. Provisional Patent Application No. 62/870,559 filed on Jul. 3, 2019 and the benefit of U.S. Provisional Patent Application No. 63/019,457 filed on May 4, 2020, the entire contents of which are all hereby incorporated herein by reference.

### TECHNICAL FIELD

The present invention relates to a server application, a mobile device application, and a client/server infrastructure; and more specifically to methods, devices, and systems for managing and tracking of collection, processing, and movement of biological samples during clinical trials utilizing distributed ledger technologies.

### BACKGROUND

Clinical trials are studies undertaken to evaluate results from various scientific experiments in human subjects. These studies are often related to the development of a new or improved pharmaceutical drug. Various types of samples, leading to the generation of data, from human test subjects are collected over the course of clinical research which generally encompasses phase 1, phase 2, and phase 3 clinical trials. The main goal of clinical research is to determine if the drug is effective, and if it is safe for humans to use based on data collected during the trials. Ultimately, this data from clinical trials may be used to determine whether the drug is approvable by health authorities and can be subsequently be commercialized.

Sponsors (e.g., pharmaceutical companies, biotech companies, etc.) conduct the clinical trials in order to determine if their drug, or new molecular entity, is safe and effective. Within the clinical study protocol, the sponsors specify various tests on the human subjects to gather data points and measure how the drug performs. A clinical study site (i.e., a facility at which trial subjects are tested) performs various tests on study subjects, including assessments of safety (e.g., adverse event reporting and safety lab samples) and efficacy (e.g., heart rate, blood pressure, or many other mechanism or indication specific tests). A bioanalytical laboratory is a specialized center that receives and analyzes biological samples from the clinical site. Biological samples (e.g., blood samples, urine samples, cerebral spinal fluid, tissue samples, or any other similar biological sample) and data (safety and efficacy measures) can be collected from tens, hundreds or thousands of test subjects, sometimes across several months or years. Each sample may be used for multiple purposes (e.g., the same blood sample used for drug concentration and circulating biomarker) and lead to various data points that a sponsor may use in determining the viability of the new drug. Additionally, the test subjects for a given clinical trial may be distributed across the world, and the individual samples may be collected at multiple and distinct clinical sites. When a given sample is collected at a clinical site, that sample may be shipped to a specialized center (e.g., central laboratory and then a bioanalytical laboratory) for sample analysis. Many different specialized centers may be utilized for a given clinical trial.

This wide range of clinical sites and specialized centers for sample analysis may lead to problems with maintaining a proper chain of custody (CoC) of the samples. Throughout the clinical trial process, the data associated with the samples can be corrupted (e.g., because of improper data entry, use of paper records, sample switching, misprinted sample tube labels, etc.) as the CoC degrades. Improper data entry may occur because the captured data are incorrectly transposed into a system (e.g., a spreadsheet), or because different clinical sites and/or bioanalytical laboratories have different formats for data entry that may not be compatible with one another. The clinical site, the central laboratory and bioanalytical laboratory each keep CoC records discrete from one another in different databases. In other words, the data from different sites and/or bioanalytical laboratories may have been entered in different formats that cannot be combined without losing the data. This creates uncertainty in the data, because a sponsor may be unable to determine which data points are pristine and accurate, and which data points have been corrupted. The uncertainty can lead to decreased accuracy in decision making on the part of the sponsor, who then may be unable to confidently rely on the data.

Additionally, health authorities (e.g., regulatory agencies like the FDA) need to review the data to arrive at a conclusion of the risk/benefit, and optimal dose, in order to approve the drug before it can go to market. Any uncertainty in data may impact the health authorities' ability to approve the drug. Minimally, it can lead to longer approval times from the health authority, or the health authority may require a clinical study to be repeated because the data was unusable. Either way, this comes at a cost of time and money, and can delay the drug being available to the public. Paper logs and records for large clinical trials also create storage and misplacement issues, specifically for audits initiated after time has passed.

Accordingly, a need exists to better facilitate the management and tracking of collection, processing, and movement of biological samples during clinical trials.

### SUMMARY

The presently disclosed subject matter is directed toward methods, devices, and systems for solving the problem of management and tracking of collection, processing, and movement of biological samples during clinical trials. These methods, devices, and systems provide for electronic collection of sample level data (e.g., time/date/subject ID) and blockchain technologies preventing fraud and cyber security breaches.

According to one embodiment, a method is implemented on one or more servers. The method includes receiving first biological sample data from a first mobile device. The first mobile device includes a camera for capturing at least a portion of the first biological sample data and a first graphical user interface (GUI) configured for displaying the first biological sample data. The method further includes storing the first biological sample data within a distributed ledger. The distributed ledger may include at least one block chain.

In some embodiments, the method may further include retrieving the first biological sample data from the distributed ledger, and transmitting the first biological sample data to a GUI on a second mobile device. The method may also include receiving second biological sample data from the second mobile device, and storing the second biological sample data within the distributed ledger.

In some embodiments, the method may further include retrieving the first biological sample data and the second biological sample data from the distributed ledger, and transmitting the first biological sample data and the second biological sample data to a third GUI on a third mobile device. The third GUI may include a dashboard. The dashboard may include a visualization tool for graphically displaying the first biological sample data and the second biological sample data, and a filtering tool for filtering the first biological sample data and the second biological sample data.

In some embodiments, the first biological sample data may include a first unique sample identifier associated with a first biological sample collected from a first clinical trial participant. The first unique sample identifier may be a barcode, a quick response (QR) code, and/or the like.

In some embodiments, the first biological sample data may further include a first clinical trial subject identifier associated with the first clinical trial participant. The first biological sample data may also include a timestamp (that may also include a date stamp) associated with a first collection time of the first biological sample. The first biological sample data may further include a first location identifier associated with a first capture location of the first biological sample. The first capture location may be a clinical site, a central laboratory, and/or a bioanalytical laboratory.

In some embodiments, the server(s) may be a virtual server(s). The virtual server may be hosted in a cloud computing environment.

In some embodiments, the first biological sample data may be received via a first secure web portal provided for the first mobile device.

In some embodiments, the first GUI may be provided by a first application specific program executing instructions on the first mobile device. The first mobile device may be a smart phone, a tablet, or the like. The first application specific program may be an iOS® app, an Android® OS app, or the like.

According to another embodiment, a server includes a memory, a database, and a processor configured for providing a method of management and tracking of collection, processing, and movement of samples during clinical trials. The method includes receiving first biological sample data from a first mobile device. The first mobile device includes a camera for capturing at least a portion of the first biological sample data and a first GUI configured for displaying the first biological sample data. The method further includes storing the first biological sample data within a distributed ledger. The distributed ledger may include at least one block chain.

According to another embodiment, a non-transitory computer readable medium includes a plurality of machine-readable instructions. The machine-readable instructions when executed by one or more processors of a server are adapted to cause the server to perform a method of management and tracking of collection, processing, and movement of samples during clinical trials. The method includes receiving first biological sample data from a first mobile device. The first mobile device includes a camera for capturing at least a portion of the first biological sample data and a first GUI configured for displaying the first biological sample data. The method further includes storing the first biological sample data within a distributed ledger. The distributed ledger may include at least one block chain.

According to another embodiment, a system includes at least one server and a plurality of mobile devices. The mobile devices are configured to communicate with the server over a network. The server includes a memory, a database, and a processor configured for providing a method of managing and tracking of collection, processing, and movement of samples during clinical trials. The method includes receiving first biological sample data from a first mobile device. At least one mobile device includes a camera for capturing at least a portion of the first biological sample data and a first GUI configured for displaying the first biological sample data. The method further includes storing the first biological sample data within a distributed ledger. The distributed ledger may include at least one block chain.

According to another embodiment, a method is disclosed for providing management and tracking of collection, processing, and movement of a plurality of biological samples during clinical trials. The method is implemented on at least one server and includes identifying interface requirements for a set of services to be implemented between service-oriented architecture (SOA) front-end components and SOA back-end components. At least one of the SOA back-end components is configured for communicating with at least one biomarker laboratory information management system (LIMS). At least one of the SOA back-end components is configured for communicating with at least one central LIMS. At least one of the SOA back-end components is configured for communicating with at least one pharmacokinetic LIMS. At least one of the SOA back-end components is configured for communicating with at least one sample repository LIMS. At least one of the SOA front-end components is configured for communicating with mobile device applications configured to capture biological sample data. At least one of the SOA front-end components is configured for communicating with computing device applications configured to manage and track the biological sample data. The SOA front-end components are operable to be combined with the SOA back-end components to form an operable SOA solution.

According to another embodiment, a server is disclosed for providing management and tracking of collection, processing, and movement of a plurality of biological samples during clinical trials. The server includes at least one memory and at least one processor. The processor(s) is (are) configured for identifying interface requirements for a set of services to be implemented between SOA front-end components and SOA back-end components. At least one of the SOA back-end components is configured for communicating with at least one biomarker laboratory LIMS. At least one of the SOA back-end components is configured for communicating with at least one central laboratory LIMS. At least one of the SOA back-end components is configured for communicating with at least one pharmacokinetic laboratory LIMS. At least one of the SOA back-end components is configured for communicating with at least one sample repository LIMS. At least one of the SOA front-end components is configured for communicating with mobile device applications configured to capture biological sample data. At least one of the SOA front-end components is configured for communicating with computing device applications configured to manage and track the biological sample data. The SOA front-end components are operable to be combined with the SOA back-end components to form an operable SOA solution.

According to another embodiment, a non-transitory computer-readable storage medium is disclosed for providing management and tracking of collection, processing, and movement of a plurality of biological samples during clinical trials. The non-transitory computer-readable storage medium storing instructions to be implemented on at least one computing device including at least one processor, the instructions when executed by the at processor(s) cause the computing device(s) to identify interface requirements for a set of services to be implemented between SOA front-end components and SOA back-end components. At least one of the SOA back-end components is configured for communicating with at least one biomarker laboratory LIMS. At least one of the SOA back-end components is configured for communicating with at least one central laboratory LIMS. At least one of the SOA back-end components is configured for communicating with at least one pharmacokinetic laboratory LIMS. At least one of the SOA back-end components is configured for communicating with at least one sample repository LIMS. At least one of the SOA front-end components is configured for communicating with mobile device applications configured to capture biological sample data. At least one of the SOA front-end components is configured for communicating with computing device applications configured to manage and track the biological sample data. The SOA front-end components are operable to be combined with the SOA back-end components to form an operable SOA solution.

### BRIEF DESCRIPTION OF THE DRAWINGS

The present embodiments are illustrated by way of example and are not intended to be limited by the figures of the accompanying drawings. In the drawings:

FIG. 1A depicts a block diagram illustrating a flow of data from a single subject in a clinical trial to a sponsor in accordance with embodiments of the present disclosure.

FIG. 1B depicts a block diagram illustrating the flow of data from multiple subjects in a clinical trial to a sponsor in accordance with embodiments of the present disclosure.

FIG. 2 depicts a block diagram illustrating a sample distributive ledger recording data from a single subject in a clinical trial in accordance with embodiments of the present disclosure.

FIG. 3 depicts a block diagram illustrating a network connecting clinical sites and/or bioanalytical laboratories and a sponsor in a clinical trial in accordance with embodiments of the present disclosure.

FIG. 4 depicts a block diagram illustrating the dashboard in accordance with embodiments of the present disclosure.

FIG. 5 depicts a block diagram illustrating the flow of data between a sponsor and one or more health authorities in accordance with embodiments of the present disclosure.

FIG. 6 depicts a system illustrating a client/server architecture for management and tracking of collection, processing, and movement of biological samples during clinical trials in accordance with embodiments of the present disclosure.

FIG. 7 depicts an example block diagram illustrating a server of FIG. 6 in accordance with embodiments of the present disclosure.

FIG. 8 depicts an example block diagram illustrating a mobile device of FIG. 6 in accordance with embodiments of the present disclosure.

FIG. 9 depicts an example block diagram illustrating a personal computer (PC) of FIG. 6 in accordance with embodiments of the present disclosure.

FIG. 10A depicts illustrating a manual workflow as currently in use within clinical trials in accordance with embodiments of the present disclosure.

FIG. 10B depicts flow diagram illustrating an automated workflow in accordance with embodiments of the present disclosure.

FIG. 11 depicts a block diagram illustrating a life cycle including various physical locations the biological samples may travel during the clinical trial in accordance with embodiments of the present disclosure.

FIG. 12 depicts a block diagram illustrating a service-oriented architecture (SOA) for management and tracking of collection, processing, and movement of biological samples during clinical trials in accordance with embodiments of the present disclosure.

FIG. 13 depicts diagram illustrating a system overview in accordance with embodiments of the present disclosure.

### DETAILED DESCRIPTION

The following description and drawings are illustrative and are not to be construed as limiting. Numerous specific details are described to provide a thorough understanding of the disclosure. However, in certain instances, well-known or conventional details are not described in order to avoid obscuring the description.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

Disclosed herein are TruLab's methods, devices, and systems for solving the problem of management and tracking of collection, processing, and movement of samples during clinical trials. In general, the disclosure relates to a method of tracking samples and associated sample-level data (e.g., subject ID, collection timestamp, custody logs, etc.) in order to ensure the integrity of the data. Maintaining pristine, data may allow for more reliable and faster decision making when reviewing results from clinical trials. It will also eliminate the need for paper requisition forms at the clinical site.

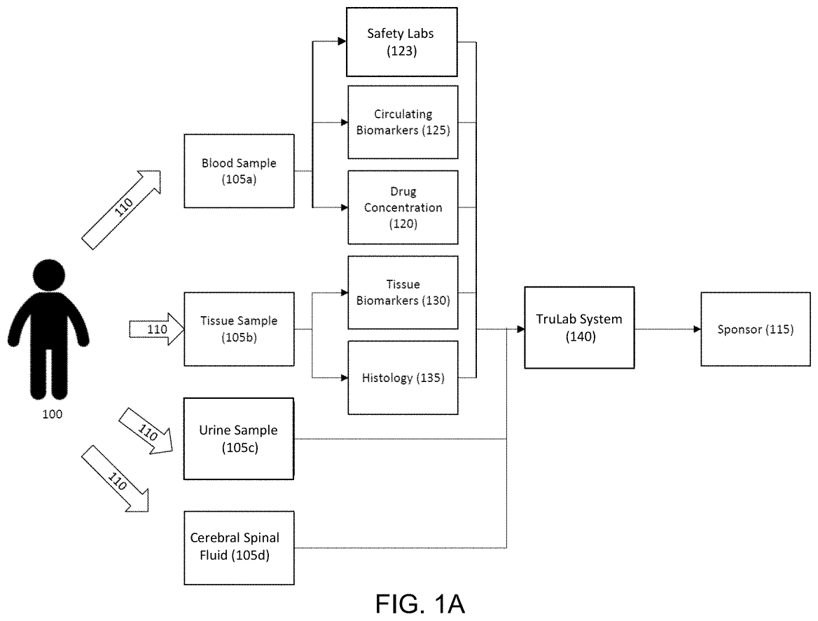

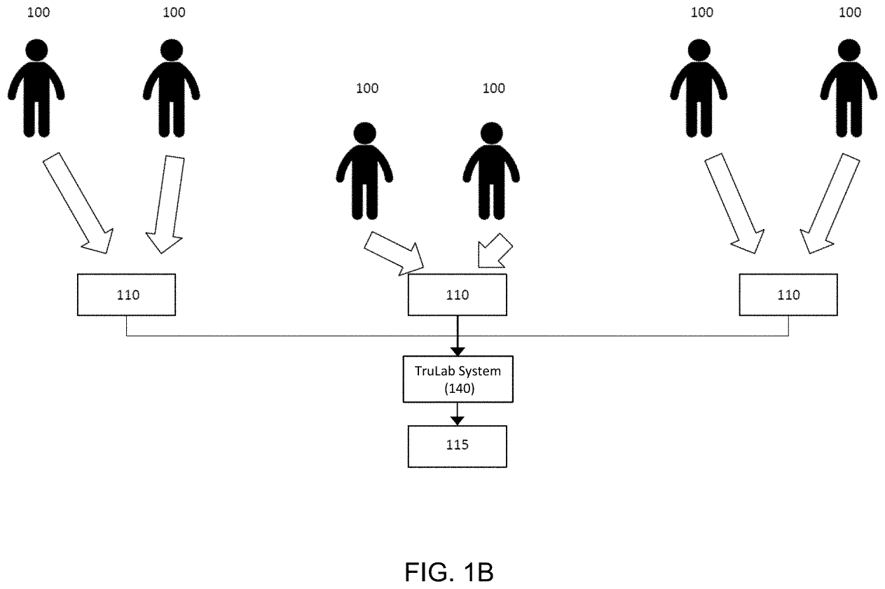

FIG. 1A depicts a block diagram illustrating a flow of data from a single subject in a clinical trial to a sponsor. Human test subjects (i.e. participants) 100 volunteer for clinical trials so that sponsors (e.g., pharmaceutical companies) can collect data on how a new product (e.g., a new molecular entity) performs. While enrolled in the trial, the test subjects 100 provide biological sample(s) 105 to clinical sites and/or bioanalytical laboratories (e.g., testing sites). These samples can include blood samples 105a, tissue samples 105b, urine samples 105c, cerebrospinal fluid 105d or any other testable sample. Clinical sites, central laboratories and/or bioanalytical laboratories 110 generate sample-level data and report the data back via the TruLab system 140 to the sponsors 115.

In the illustrated embodiment, a variety of tests can be run on a single sample (e.g., a blood sample 105a). This can include testing for drug concentration 120 in the blood, testing for circulating biomarkers 125, or any other similar test. Similarly, testing for tissue markers 130 and conducting a histology 135 may be performed for tissue samples. After the tests are completed, the sample-level data associated with the tests are transferred to the sponsor 115 via the TruLab system 140. The sponsor 115 uses this data in their analysis of results in order to determine how the drug is performing. Additionally, the biological samples may be sent to a long-term storage repository.

FIG. 1B depicts a block diagram illustrating the flow of data from multiple subjects in a clinical trial to a sponsor. Multiple test subjects 100 may be associated with each clinical site and/or bioanalytical laboratory 110, and multiple clinical sites and/or bioanalytical laboratories 110 may be associated with each sponsor 115. In other words, a sponsor 115 receives data from a variety of sources. With each additional test run, the volume of data increases, as well as the need to ensure that all of the data within a chain (e.g., all data collected from a single subject 100) remains accurate. In order to maintain the integrity of the data, and the overall validity of the clinical trial, proper chain of custody (CoC) of all data is necessary.

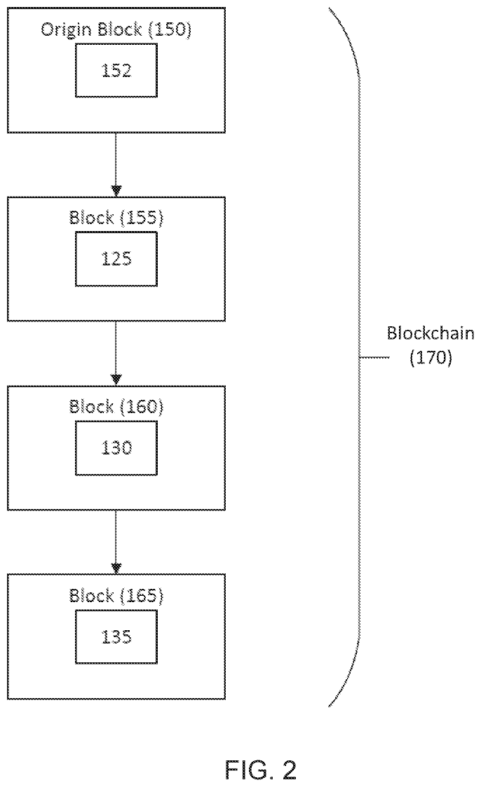

FIG. 2 depicts a block diagram illustrating a sample distributive ledger recording data from a single subject in a clinical trial. A distributive ledger technology (e.g., block chain) is utilized by the sponsor and the clinical sites and/or bioanalytical laboratories in order to maintain accurate data collection. The clinical sites and/or bioanalytical laboratories creates an origin block 150 for a subject 100 new to the clinical trial. Samples 105 collected from the subject 100, or tests run on those samples, are recorded in subsequent blocks 155, 160, 165, etc. Each subsequent block 155, 160, 165 would include the information specifically associated with the clinical sites and/or bioanalytical laboratories' action (e.g., drawing blood, testing the blood, etc.) and would be linked to the previous blocks 150, 155, 160. In other words, new block 165 would link to the preceding block 160 to insure CoC, and would include new data unique to block 165. After the validity of each new block is tested (e.g., through testing by other nodes on a computer network), the block 155 is added to the block chain 170. The block chain 170 creates an overall ledger associated with each subject 100.

Since the validity (e.g., accuracy) of each new block 155 is verified prior to being added to the block chain 170, the data collected by the clinical sites and/or bioanalytical laboratories 110 becomes substantially immutable. Additionally, the data cannot be changed or otherwise altered after it has been added to the block chain 170. This creates a reliable CoC that sponsors 115 can rely on when analyzing the data.

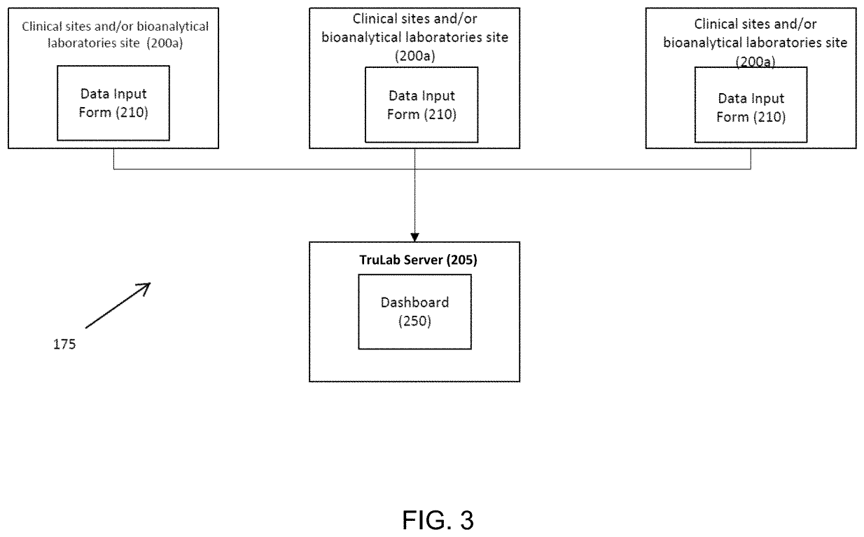

FIG. 3 depicts a block diagram illustrating a network connecting clinical sites and/or bioanalytical laboratories and a TruLab server in a clinical trial. Additional sites (not shown in FIG. 3) include repositories where the biological samples remain after the clinical trial. Sponsors 115 and clinical sites and/or bioanalytical laboratories 110 are connected over a network 175 in order to upload and track the data collected for the clinical trial. The network 175 includes clinical sites and/or bioanalytical laboratories site 200a and a TruLab server 205. The clinical sites and/or bioanalytical laboratories 200a and TruLab server 205 are connected via the block chain 170. The final repository may also be connected by the block chain 170 (not shown in FIG. 3). Each clinical sites and/or bioanalytical laboratories site 200 communicates directly with the TruLab server 205, but the individual clinical sites and/or bioanalytical laboratories sites 200 do not communicate with one another. In the illustrated embodiment, the clinical sites and/or bioanalytical laboratories site 200 includes a data input form 210 that allows the clinical sites and/or bioanalytical laboratories 110 to input data associated with each subject 100. In some embodiments, the data input form 210 may be standard (e.g., may utilize Food and Drug Administration (FDA) approved C-Disk format) across all clinical sites and/or bioanalytical laboratories sites 200. This creates consistent data entry across all clinical sites and/or bioanalytical laboratories 110.

After each block 155 is verified, the data is transferred to and stored on the TruLab server. In some embodiments, the data may be stored in a physical location (e.g., a hard drive, a server, etc.). In other embodiments, the data may be stored in the cloud upon reaching the TruLab server 205. In some embodiments, the sponsor 115 may be an administrator of the data collected during the clinical trial, and may be responsible for maintaining the server and/or cloud.

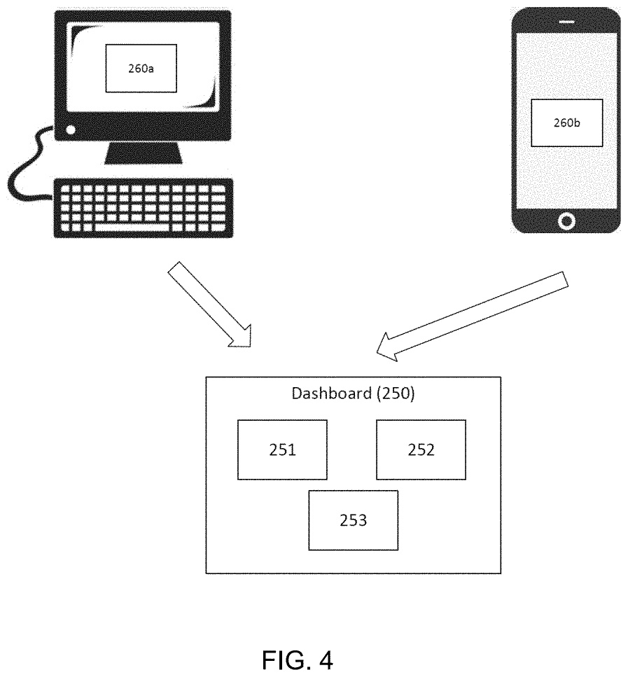

FIG. 4 depicts a block diagram illustrating the dashboard. The TruLab server 205 includes a dashboard 250 that allows the sponsor 115 to interact and visualize the vast amounts of data collected throughout the clinical trial and logged by the clinical sites, central laboratory, and/or bioanalytical laboratories 110. The dashboard 250 complies all of the saved data, and allows the sponsor 115 to interact with the data. The dashboard 250 includes a filtering tool 251 and a visualizing tool 252. The filtering tool 251 allows the sponsor 115 to customize how the data is sorted. The visualizing tool 252 allows the sponsor 115 to graphically present the data. In the illustrated embodiment, the dashboard 250 also includes a security tool 253 that allows the sponsor to control other party's access to the dashboard 250.

In the illustrated embodiment, the dashboard 250 is linked in real time to the clinical sites and/or bioanalytical laboratories site(s) 200. In other words, changes made on the clinical sites and/or bioanalytical laboratories site 200 (e.g., entering new data points in the block chain) are instantly (or substantially instantly) transferred to the TruLab server 205, and made available on the dashboard 250. Block chain 170 allows the data to be instantly available to the network 175 after being verified, and the dashboard 250 continuously updates in order to mine newly entered biological sample collection and tracking information. This allows sponsors via the TruLab server 205 to analyze trends associated with the sample collection and tracking in real time, as opposed to waiting weeks or months for the data to be recorded by the clinical sites and/or bioanalytical laboratories 110 and transferred to the sponsors 115.

The dashboard 250 is available to the sponsor 115 via the TruLab server 205 via an electronic device 260. In some embodiments, the electronic device may be a computer 260a (e.g., a laptop computer, a desktop computer, etc.). In other embodiments, the electronic device may be a portable device 260b (e.g., a mobile phone, a tablet, etc.). In some embodiments, the dashboard 250 may be web-based. In other embodiments, the dashboard 250 may be available through downloadable content (e.g., a program, an application, etc.). In any embodiment, sponsors 115 can interact with the dashboard 250 via graphical user interfaces (GUIs). The dashboard 250 on the computer 260a may have a substantially similar visual aesthetic as the dashboard 250 on the portable device 260b.

In the illustrated embodiment, the dashboard 250 is used to more easily sort through and interact with the data collected by the clinical sites and/or bioanalytical laboratories 110. For example, the sponsors via the TruLab server 205 may be able to run reports and use graphs to illustrate trends in the data. This may include being able to track the location of samples, generate missing sample reports, track actual number of samples collected compared to the planned amount, and view all samples associated with a specific subject.

The dashboard 250 via the TruLab server 205 may also use artificial intelligence (AI) (e.g., machine learning) to analyze the data for trends and outliers. AI may be able to identify trends quicker than humans are able to, and may also be able to quickly identify data points that are statistical anomalies with respect to the overall data set. Although by the nature of using a block chain 170, the data should be accurate, the sponsor 115 can flag the data point and verify with the clinical sites and/or bioanalytical laboratories 110 that the value is correct (i.e., that the point is in fact an outlier, and not a mistake). If the data point is correct, the sponsor 115 can attempt to determine the cause of the outlier, and if any other steps need to be taken.

Approval from health authorities or monitors is required as the clinical trial progresses. Global health authorities (e.g., the FDA) and sometimes, independent data monitoring committees (IDMC), may need to review the biological sample data and findings from the study. Since the dashboard 250 uses block chain 170 to record the sample collection and tracking data, these independent bodies can quickly assess whether the data generated from the sample collection is accurate. This is helpful for agencies like the FDA because they know that the data from the clinical trial is accurate, and may more confidently make decisions on approving or rejecting the drug. Additionally, this is beneficial for the sponsor because they know that they will not be required to repeat tests. This will save time and money correcting human error on otherwise approvable drugs. Capturing sample-level data such as the time, date, subject ID, and associated meta-data will provide detailed audit trails as required by health authorities such as the FDA, and which may prevent fraud. In addition, storing the data in blockchain will ensure data integrity, creating another level of trust for the sponsor, the FDA and other regulators. In addition, the databases may be backed up every 24 hours, and each database may be hashed and placed on a distributed ledger.

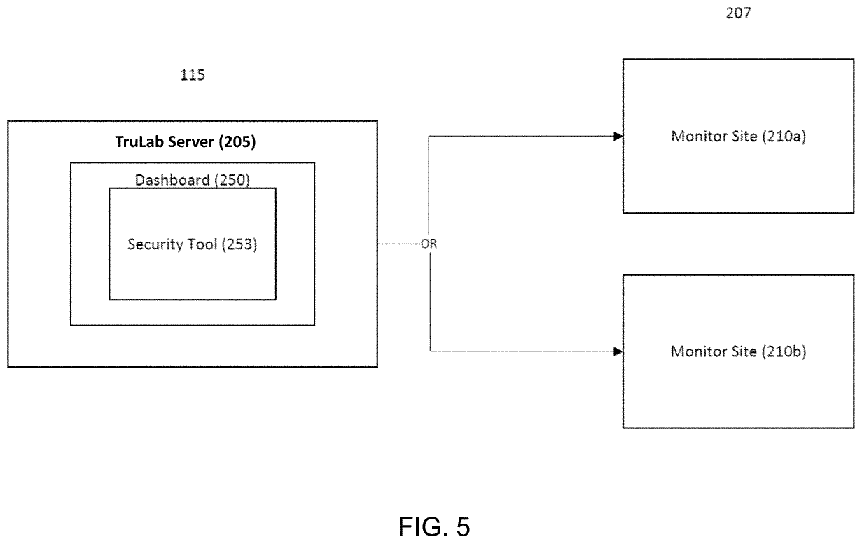

FIG. 5 depicts a block diagram illustrating the flow of data between a sponsor and one or more health authorities

In some embodiments, the sponsor 115 may give the monitors unlimited access of the dashboard 250 through the security tool 253. This allows the monitors to view substantially the same information as the sponsor. This information includes sample data level such as collection time and date, subject ID, freezer check-in times, and aliquot data. The monitors may be able to regulate user compliance over the course of the study. In other embodiments, the sponsor 115 may give the monitors controlled access to the dashboard 250 while the clinical trial is progressing. This allows the monitors to observe the study generally, without being able to use some features of the dashboard 250.

The monitors may have substantially the same program or application (i.e., monitor site 210a) as the sponsor 115, with the sponsor 115 able to regulate the monitor's access. The sponsor 115 may be able to selectively grant or revoke the monitor's access to the dashboard 250 at various points during the clinical trial. The sponsor 115 may also be able to selectively change the level of access of the monitors during the study. For example, this may be done by providing monitors various access codes that expire after a predetermined period of time. Monitors can also be given access to the dashboard 250, and the sponsors 115 can selectively access to various features, and deny access to others. The monitor site 210b may also be different than the TruLab server 205 site and accessible through a separate program or application. The monitor site 210 may have fewer features than the TruLab server 205, so that the sponsor 115 may not need to actively control the monitor site 210.

In other embodiments, the dashboard 250 could be used with other applications. Other types of data can be collected and stored on the block chain 170, and visualized on the dashboard 250. A sponsor, or similar manager of the dashboard 250, can use the features to analyze the data using similar techniques as have been already described above.

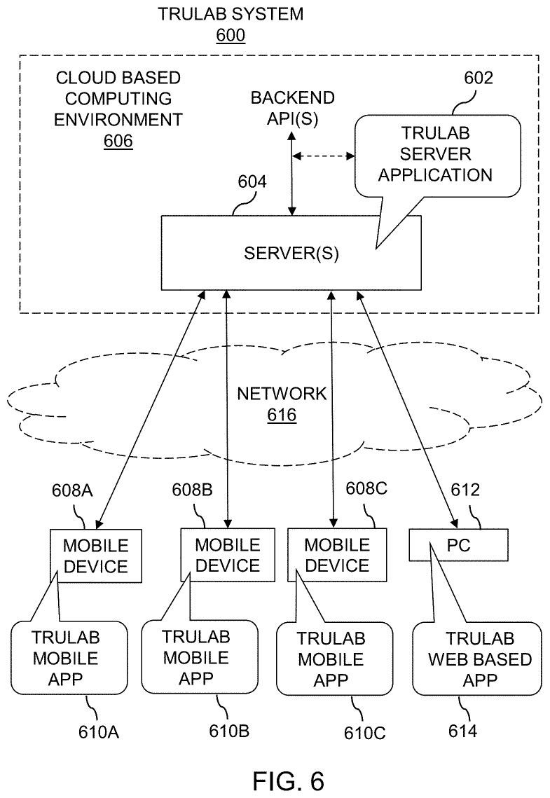

FIG. 6 depicts a TruLab system 600 implemented as another embodiment. The TruLab system 600 is a client/server architecture that includes a TruLab server application 602 hosted on a server 604. The server 604 is resident in a cloud based computing environment 606. In other embodiments, the server 604 may be housed at a central lab or other datacenter. The server 604 communicates with a plurality of mobile devices 608A-C, hosting a plurality of TruLab mobile apps 610A-C. The mobile devices 608A-C may be smart phones, tablets, or the like. The server 604 also communicates with a personal computer (PC) 612, hosting a TruLab web based app 614. In some embodiments, a laptop or tablet may be used instead of the PC 612 to host the TruLab web based app 614. The TruLab mobile apps 210A-C and the TruLab web based app 612 communicate with the TruLab server application 602 over a network 616. The network 616 may be any type or combination of wired, wireless, and/or optical networks. The network 616 may include the Internet. Additionally, the TruLab server application 602 may communicate using one or more backend application programming interfaces (APIs) to one or more other systems associated with one or more clinical trials. The backend APIs may communicate within the cloud based computing environment 606 or over the network 616.

The server 604 with the TruLab server application 102 may perform any of the methods described in the summary and the claims. The TruLab server application 102 transforms the server 104 from a generic computer function into a machine for solving the problem of management and tracking of collection, processing, and movement of biological samples during clinical trials.

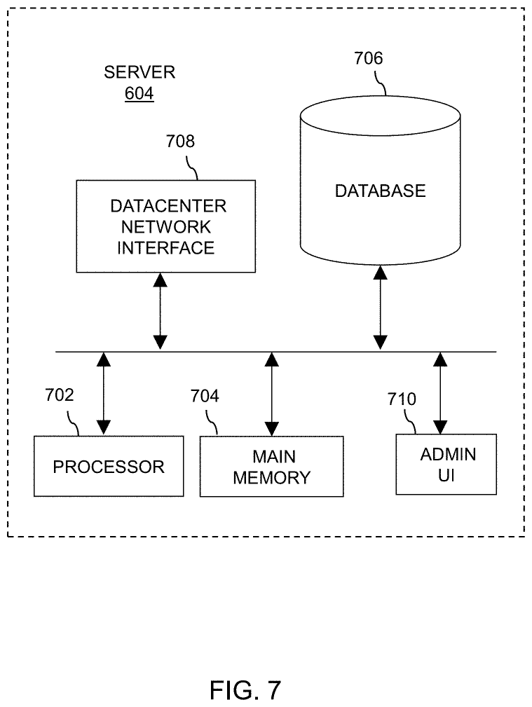

FIG. 7 illustrates an example block diagram of the server 604 of FIG. 6. The server 604 includes at least one processor 702, a main memory 704, a storage memory (e.g. database) 706, a datacenter network interface 708, and an administration user interface (UI) 710. The server 604 may be configured to host an Ubuntu® server or the like. In some embodiments the Ubuntu® server may be distributed over a plurality of hardware servers using hypervisor technology.

The processor 702 may be a multi-core server class processor suitable for hardware virtualization. The processor may support at least a 64-bit architecture and a single instruction multiple data (SIMD) instruction set. The main memory 704 may include a combination of volatile memory (e.g. random access memory) and non-volatile memory (e.g. flash memory). The database 706 may include one or more hard drives. The database 706 may also be configured to store at least a portion of a distributed ledger that includes at least one block chain.

The datacenter network interface 708 may provide one or more high-speed communication ports to data center switches, routers, and/or network storage appliances. The datacenter network interface 708 may include high-speed optical Ethernet, InfiniB and (IB), Internet Small Computer System Interface (iSCSI), and/or Fibre Channel interfaces. The administration UI may support local and/or remote configuration of the server 604 by a datacenter administrator.

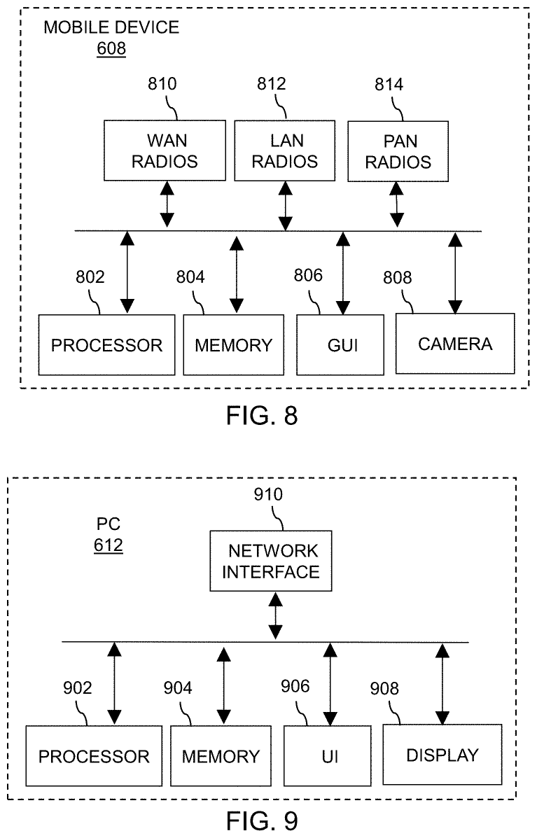

FIG. 8 illustrates an example block diagram of the mobile device 608 of FIG. 6. The mobile device 608 may include at least a processor 802, a memory 804, a GUI 806, a camera 808, wide area network (WAN) radios 810, local area network (LAN) radios 812, and personal area network (PAN) radios 814. In some embodiments, the mobile device 608 may be an iPhone® or an iPad®, using iOS® as an operating system (OS). In other embodiments, the mobile device 608 may be an Android® OS device. In certain embodiments, the camera 808 may be implemented as a separate barcode scanner, QR scanner, or the like. In this embodiment, the mobile device 608 may connect the camera 808 to the other mobile device components via a Blue Bluetooth® link, universal serial bus (USB) link, or the like.

In some embodiments, the processor 802 may be a mobile processor such as the Qualcomm® Snapdragon™ mobile processor. The memory 804 may include a combination of volatile memory (e.g. random access memory) and non-volatile memory (e.g. flash memory). The memory 804 may be partially integrated with the processor 802. The GUI 706 may be integrated such as a touchpad display. The WAN radios 810 may include 2G, 3G, 4G, and/or 5G technologies. The LAN radios 812 may include Wi-Fi technologies such as 802.11a, 802.11b/g/n, 802.11ac, and/or 802.11ax circuitry. The PAN radios 814 may include Bluetooth® technologies.

FIG. 9 illustrates an example block diagram of the PC 612 of FIG. 6. The PC 612 may include at least one processor 902, at least one memory 904, a user interface (UI) 906, at least one display 908, and a network interface 910. In certain embodiments, the PC 612 may be a workstation class computing device. The processor 902 may be an Intel core i9-10900K desktop processor or the like. The memory 904 may include a combination of volatile memory (e.g. random access memory) and non-volatile memory (e.g. flash memory). The memory 904 may be partially integrated with the processor 902. The UI 910 may include a keyboard. The UI 910 may also include a mouse, at touchpad, or the like. In certain embodiments, the UI 910 may be integrated with the display 906. The display 908 may be a separate display or may be integrated with the other components (e.g. laptop).

The PC 612 may include an operating system (OS) to run the TruLab web based app 614 as shown in FIG. 6. The operating system (OS) may be a Windows® OS, a Macintosh® OS, a Linux® OS, or the like. The network interface 910 may be a wired Ethernet interface or a Wi-Fi interface. The PC 612 may be configured to access remote memory (e.g. network storage and/or cloud storage) via the network interface 910. In other embodiments, the PC 612 may be a smart TV configured and the TruLab web based app 612 may be configured as a smart TV app.

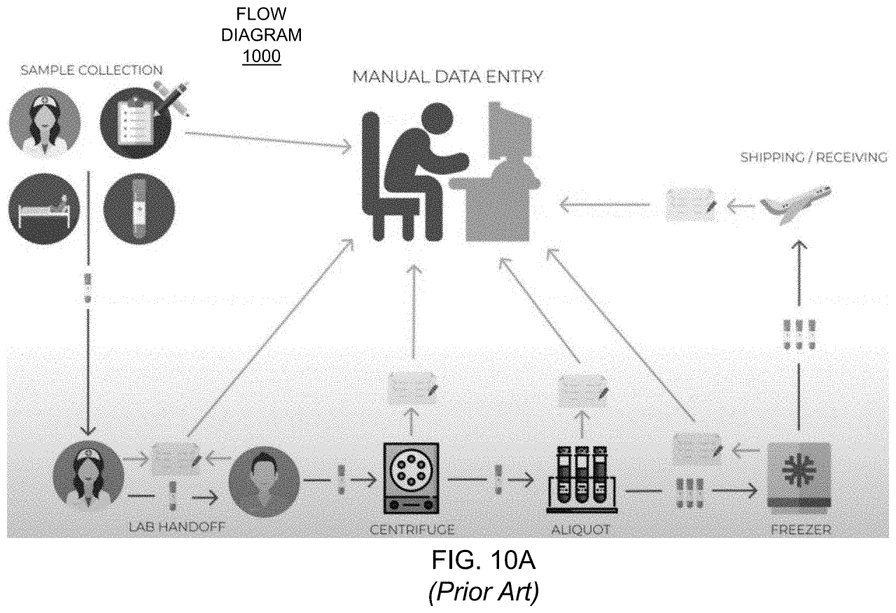

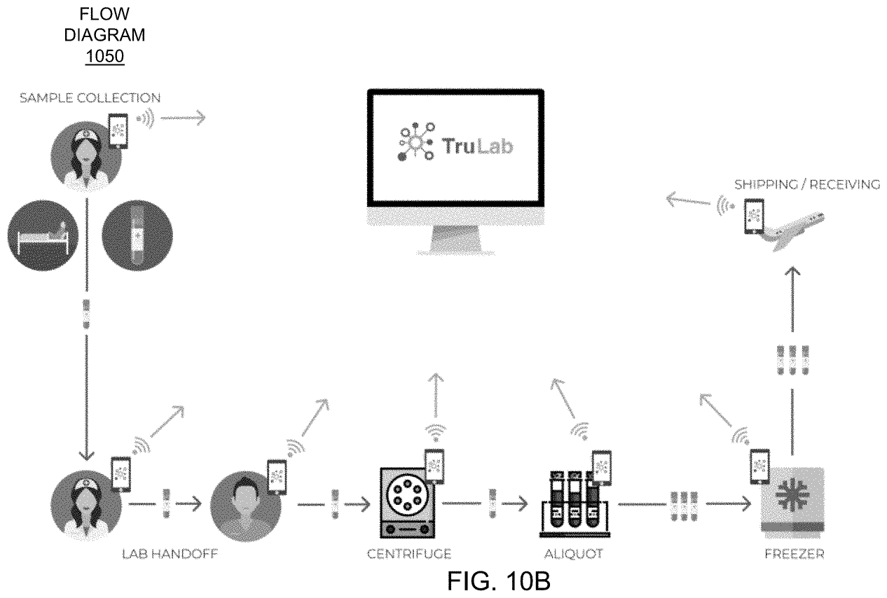

Further illustrating the capabilities of the TruLab system 600, the current mostly paper process is reviewed. FIG. 10A depicts a flow diagram 1000 of a manual data entry process including (1) sample collection, (2) chain of custody handoff, (3) centrifuge processing, (4) division to aliquots, (5) freezer storage, and (6) shipping/receiving of the biological samples. The sample collection starts when a clinical study staff member takes a biological sample from a subject (or a patient). Next, the clinical study staff member handwrites a subject identification (ID), a visit number, and a time and date on a test tube containing the biological sample. The clinical site staff then completes a paper case report form (CRF) with the requisite information. Next, the site staff member walks the biological sample down to a lab and hands off the biological sample to a lab technician. The clinical study staff and lab technicians will sign into a paper log to document that the handoff process took place. The lab technician will then process the biological sample in a centrifuge. Next the lab technician will document the centrifuge processing in a separate paper log including a temperature, a speed, and start and stop times. The lab technician may then aliquot the biological sample into additional test tubes and then document into yet another paper form or log. Next, the lab technician will walk the biological sample to a freezer for storage and document the transfer in another paper log. When ready for shipment, the biological sample will be removed from the freezer and prepared for shipping. This removal is then documented in another paper log and a separate paper shipping log. Some of this paper trail (i.e. paper logs and labels) will remain in paper and other parts may be manually entered into an electronic data capture (EDC) system or other database system.

FIG. 10B depicts a flow diagram 1050 illustrating how the TruLab system 600 (including the TruLab server application 602 and the TruLab mobile app 610) may eliminate many if not all of the manual entry steps. The TruLab system 600 eliminates paper requisition forms and paper logs that were previously described in FIG. 10A at each step by allowing the clinical study staff to leverage the TruLab mobile app 610 and the TruLab server application 602. Using barcode technology associated with the mobile device 608 (including the camera 808), the TruLab mobile app 610 eliminates this previously described manual data capture and entry that takes up valuable bandwidth of the study staff. The TruLab mobile app 610 may be installed on a clinical site staff's personal smartphone or on a mobile device provided by the clinical trial.

The TruLab mobile app 610 provides a home screen that includes selections for “sample collection”, “digital logbook”, “shipping/receiving”, and “screening”. These selections allow clinical site staff to barcode scan biological sample test tubes at the point of collection. The digital logbook replaces the previously described paper logs. The shipping/receiving selection allows clinical site staff to document the shipping process. The entire process is secured in block chain technology within a distributed ledger associated with the TruLab server application 602.

Various GUI screens facilitate the sample collection process within the TruLab mobile app 610. When using the “sample collection” selection, a protocol is selected from a drop-down menu. Next the subject ID is selected from a drop down menu or the subject's wristband may be scanned. Once identification is made, a sampling schedule for the subject is displayed within the TruLab mobile app 610. To collect and record, a biological sample is collected from the subject and a barcode label on the test tube is scanned by the clinical site staff using the camera 808 on the mobile device 508. There may be a time delay from when the biological sample was collected and when the clinical site staff can complete the scan. The TruLab mobile app 610 allows the time to be adjusted before submitting. Once submitted, a gamification badge is displayed on a screen showing the total number of samples the staff member has collected on their schedule. The TruLab mobile app 610 then provides the clinical site staff with a countdown (i.e. waiting period) for the time when the next sample is to be taken from the subject. The waiting period may be preprogrammed. An alert (visual and/or audible) is provided by the TruLab mobile app 610 when the time is nearing being out of window for the next scheduled collection from the subject.

The clinical site staff may use the TruLab mobile app 610 for managing and tracking multiple subjects during the clinical trial. Each staff member can view all their assigned subjects and other subjects that they may be assigned as a backup to another member of the clinical site staff. Once selecting a given subject, the subject ID, protocol number, and collection number are displayed on the top of the screen by the TruLab mobile app 610.

Various GUIs of the digital logbook selection of the TruLab mobile app 610 is provided by various GUIs. After the study staff have collected the biological samples using the sample collection features of TruLab mobile app 610, they then take the samples down to the lab for processing and storage. When arriving at the lab they go to the “digital logbook” selection and then the “custody” selection to maintain the chain-of-custody for the biological sample. This is where the clinical study staff member that collected the initial biological sample from the subject would document the handoff to the lab technician. Basically, the clinical study staff member barcode scans the test tubes they are checking into the lab and the lab technician receiving the biological samples also barcode scans in the test tubes to acknowledge receipt.

Next the lab technician may take the transferred biological samples over to a centrifuge for processing. As before the lab technician barcode scans all of the test tubes going into the centrifuge for that processing cycle. If the lab technician accidentally scans the wrong test tube, they may delete it by touching that record after all the tubes are scanned. Next, the lab technician may scan a barcode label that is affixed to the centrifuge to identify which centrifuge is being used to process the current batch of biological samples. The lab technician then enters the temperature speed and duration of that cycle of the centrifuge.

If the protocol requires for aliquoting the biological samples, the lab technician then selects the “aliquot” feature of the TruLab mobile app 610 and barcode scans the parent test tube; and then scans all of the child test tubes. The TruLab system 600 then automatically marries the parent and child test tube data into the block chain of the distributed ledger.

Various other GUIs within the digital logbook section of the TruLab mobile app 610 allow for managing the transfer of biological samples into and out of cold storage including transfer between freezers. When on or more biological samples are ready for cold storage, the lab technician (or other lab technician) would make the “freezer” selection within the TruLab mobile app 610. While in the “freezer” section, the lab technician barcode scans all the test tubes (i.e. batch scan) that are going into that particular freezer. Like the centrifuge, each freezer (and possibly each shelf within each freezer) has a unique barcode label that is scanned to document entry. The TruLab system 600 then updates the block chain of the distributed ledger with the cold storage data.

If a lab technician desires to move biological samples from one freezer to another freezer, they select the “freezer transfer” section of the TruLab mobile app 610. Next the lab technician barcode scans all the test tubes desired for transfer. Since the block chain already has a record (i.e. barcode) of the current freezer and possible shelf, only the new freezer and possible shelf need scanning. The TruLab system 600 then updates the block chain of the distributed ledger with the date and time the biological samples were moved, the original freezer, and the new freezer.

The TruLab mobile app 610 provides various GUIs for managing shipping within the shipping/receiving section. When biological samples are ready for shipping, the lab technician makes the “shipping” selection. Next, the lab technician selects the correct protocol for the biological samples. A drop-down menu then loads for all of the labs that are associated with the clinical trial. The lab technician then selects the lab and batch scans the test tubes going to that particular lab. Next, the lab technician scans the barcode for a courier's tracking label. The TruLab system 600 then updates the block chain of the distributed ledger with the shipping data for that batch of biological samples. The lab technician is then given an option to begin a different shipment.

Various GUIs illustrating clinical site remote monitoring capabilities of the TruLab web based app 614 operating on the PC 612 of FIG. 6 provides various GUIs for remote monitoring. For example, a “clinical site remote monitoring” dashboard enables clinical study sponsors (e.g. drug sponsors) and different clinical sites the ability to perform real-time monitoring (including performance of clinical site staff and lab technicians) of biological sample collection activities for a given clinical trial. Clinical research associates (CRAs), designated as liaisons between a clinical study sponsor and the different clinics may also use the dashboard.

The TruLab web based app 614 may be accessed using a web browser on the PC 612. The TruLab web based app 614 may also be accessed using any other smart device such as a smartphone, tablet, smart watch, smart television, or the like. A user monitoring the clinical trial begins by logging in with a user ID and password to access the dashboard.

The dashboard also allows a user to set up alerts based on what they desire to monitor. For example, the TruLab web based app 614 may be configured to allow the dashboard to monitor for “alerts” for “out-of-window” collections at a given clinical site for a given protocol. This real-time remote monitoring capability gives a monitor (e.g., CRA) valuable data without having to be present on site or possibly waiting weeks or months before the errors are discovered. Other alerts show how long a biological sample is out of the freezer. For example, the alert may be triggered when a biological sample has been collected but not checked into a freezer within a 60 minute window. Additional alerts may be preprogramed for sample collection kit resupply and to track missed visits to a clinical site. As disclosed earlier, the TruLab system 600 may send individual alerts to each responsible lab technician or site staff member. These individual alerts may be received via the TruLab mobile app 610. The individual alerts may also be received via a short message service (SMS) message, a multimedia messaging service (MMS) message, and/or a phone call with a computer voice generated message to the mobile device 608 of the responsible staff member. A supervisor of the responsible staff member may also receive one or more of the individual alerts.

The TruLab web based app 614 also allows for tracking biological samples collected against a given plan for an each protocol in a single view and in near real-time for the user. The user may also monitor the number of biological samples being collected for each clinical site for a given protocol.

The TruLab web based app 614 provides GUIs for setting up a new protocol within the TruLab system 600. The user (e.g. administrator and/or study coordinator) would program the schedule of assessments on the dashboard when a new protocol is being set up. In this example the user is putting in a schedule of assessments for a pharmacokinetic (PK) sample collection. The user may also enter a biological sample type and amount that you want to collect. Then the user can put in time intervals, and a time window that collection is allowable from a subject.

The TruLab web based app 614 provides GUIs for clinical site user management.

With this interface, a study coordinator and/or administrator at a given clinical site can set up and manage site staff and their associated TruLab mobile app 610. Each staff member's activities and performance may be monitored. For example, the study coordinator may monitor performance badges for a given staff member as previously described. Because, the TruLab system 600 gives real-time remote site monitoring and anomaly detection, the amount of hours a clinical research organization (CRO) may bill for CRA and central lab services is reduced.

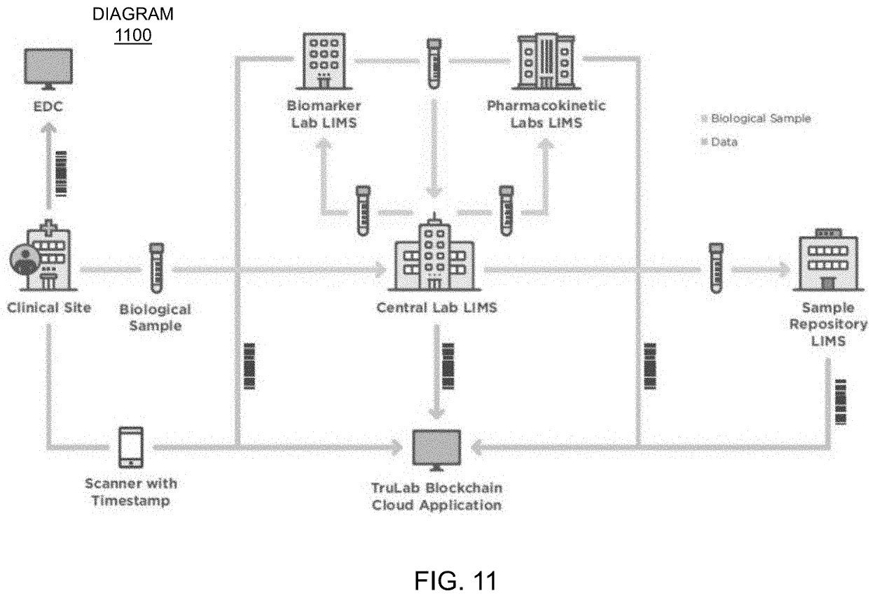

FIG. 11 depicts a block diagram illustrating a life cycle including various physical locations the biological samples may travel during the clinical trial. In support of this life cycle, the TruLab system 600 provides end-to-end sample tracking from the original clinical site to a final destination. For example, the final destination may be a central lab, a specialty lab, and/or a sample repository.

Sample kits are manufactured and sent by the central labs to the clinical sites. The test tubes included in the sample kits usually arrive at the clinical site with barcodes already attached. Most clinical sites never use the pre-applied barcodes and instead the barcodes are first utilized once the biological samples arrive back at the central lab. Then they are entered into the internal lab information system. The TruLab system 600 provides an end-to-end tracking solution via the previously described capabilities of the TruLab mobile app and the recorded data of the distributed ledger.

The drug sponsor and the central lab each get notified (including the courier's tracking number) by the TruLab server application 602 when biological samples are shipped to their next destination. The TruLab system 600 does not require each lab to use the TruLab mobile app 610 to support tracking. A given lab may use their existing system. When that barcode is scanned at the central lab and/or specialty lab their information system would push the barcode data to the TruLab system 600 via an API. When the TruLab system 600 receives that barcode data it automatically updates the distributed ledger. If an API is unavailable, the user may manually import data files provided from the given labs to update the TruLab system 600 for any given biological sample with location data.

The TruLab web based app 614 provides for end-to-end sample tracking capabilities. A GUI provides a “sample tracker” dashboard to locate any given biological sample within the clinical trial. The user selects the appropriate protocol from the dashboard and can then search by sample ID (i.e. barcode). If a barcode is not known the user may search by the subject ID, the visit number, and the date and time the biological sample was collected. The TruLab system 600 then provides the last known location for the given biological sample including the date and time it was received as shown in the GUI

The TruLab web based app 614 provides various data management capabilities including a dashboard. A data manager for a drug sponsor would use the “data management” dashboard for their data reconciliation. The TruLab system 600 supports API integration with other labs that may not be using the TruLab system 600. Via this API integration and as previously discussed in FIG. 6, the “data management” dashboard can continue to update the entire clinical trial in real-time. For labs that do not support API integration, that will not allow API integration, and/or do not have a lab information system, the TruLab system 600 has the capability to import data files. For example, comma separated values (CVS) files, Microsoft® Excel® files, and/or other spreadsheet and database files may be imported to the TruLab system 600. Data from the imported files are then mapped by the sample ID (i.e. barcode). The data manager would request that the lab include the barcode column assuming that the lab uses the barcode that exists already on each test tube. The TruLab system 600 may also export such files for these labs as needed. If the lab does not scan the barcode or does not have a lab information system, the record may be manually updated by mapping the subject ID, and the collection date and time of that sample. The data manager also has the ability via the dashboard to filter and sort. For example, the data manager, via the dashboard, may view which biological samples have been collected but not yet received by the lab, or may view how many days since the given biological sample was collected. Finally, the GUI of FIG. 20(b) provides an “audit trail” dashboard depicting all the transactions stored within the distributed ledger of the TruLab system 600. The transactions include all edits and overrides anchored within the block chain technology of the distributed ledger.

Additional capabilities of the TruLab server application 602, TruLab mobile device app 610, and the TruLab web based app 614 (i.e. TruLab system 600) may include Natural Language Processing, Artificial Intelligence (AI), and Machine Learning to automate setting up protocols. In some embodiments, the TruLab system 600 may read a portable document format (PDF) or other formatted document of a protocol and automatically populate fields such as the schedule of assessments, the dosage, the planned samples, the subject information (like ID numbering), workflow, special handling instructions, associated labs with addresses, sample temperature requirements, aliquot requirements, and/or the like.

Additional features of the TruLab system 600 may include tagging each biological sample with the appropriate informed consent, and whether including consent for the current study, future research, and genomics. It may also include the destruction date of the sample. These ‘tags’ may stay with the sample during the entire life cycle of the sample, even after the conclusion of the study.

In further embodiments, the TruLab system 600 may be adapted for providing management and tracking of collection, processing, and movement of samples for experiments other than clinical trials. For example, the TruLab system 600 may be configured for soil sample experiments, air sample experiments, water sample experiments, or the like.

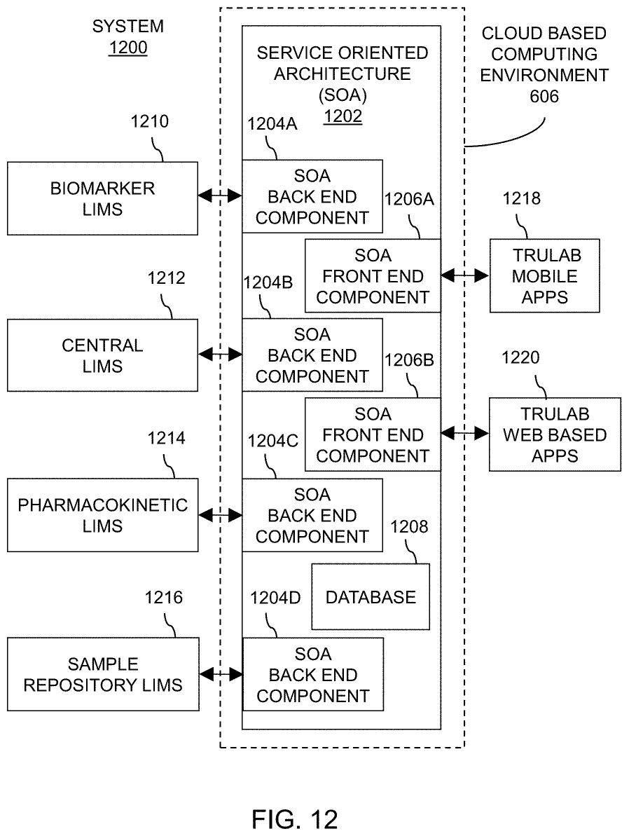

FIG. 12 depicts a block diagram for a system 1200 illustrating a service-oriented architecture (SOA) 1202. The SOA 1202 provides a collection of services, wherein the services communicate with each other. The communications may range from simple exchanges of data to two or more services coordinating one or more activities. Each service may be a function that is self-contained and well-defined. Each service may not depend on a state or context of each of the other services. The SOA 102 includes SOA back-end components 1204A-D and front-end components 1206A-B for providing management and tracking of collection, processing, and movement of biological samples during clinical trials.

The SOA 1202 is configured to provide a middleware solution that includes a method of identifying interface requirements for a set of services to be implemented between the SOA back-end components 1204A-D and SOA from-end components 1206A-B. SOA back-end component 1204A is configured for communicating with at least one biomarker laboratory LIMS 1210. SOA back-end component 1204B is configured for communicating with at least one central laboratory LIMS 1212. SOA back-end component 1204C is configured for communicating with at least one pharmacokinetic laboratory LIMS 1214. SOA back-end component 1204C is configured for communicating with at least one sample repository LIMS 1216. SOA front-end component 1206A is configured for communicating with mobile device applications configured to capture biological sample data. SOA front-end component 1206B is configured for communicating with computing device applications configured to manage and track the biological sample data. The SOA front-end components are operable to be combined with the SOA back-end components to form an operable SOA solution.

The SOA 2102 may also include a database 1208. In some embodiments, the database 1208 may be an open source database such as the MongoDB® database, the PostgreSQL® database, or the like. An additional front-end component (not shown in FIG. 12) may be configured to provide an administrator access secure web portal. The administrator access secure web portal may be configured to provide status and control of the operable SOA solution.

The SOA back-end components 1204A-D may be coupled to the LIMS 1210, LIMS 1212, LIMS 1214, and LIMS 1216 by a combination of the Internet, wide area network (WAN) interfaces, local area network (LAN) interfaces, wired interfaces, wireless interfaces, and/or optical interfaces. In some embodiments the SOA back-end components 1204A-D may also use one or more transfer protocols such as a hypertext transfer protocol (HTTP) session, an HTTP secure (HTTPS) session, a secure sockets layer (SSL) protocol session, a transport layer security (TLS) protocol session, a datagram transport layer security (DTLS) protocol session, a file transfer protocol (FTP) session, a user datagram protocol (UDP), a transport control protocol (TCP), or a remote direct memory access (RDMA) transfer protocol.

The SOA front-end components 1206A-B may be coupled to the TruLab mobile device applications 1218 and the TruLab web based application 1220 by a combination of the Internet, WAN interfaces, LAN interfaces, wired interfaces, wireless interfaces, and/or optical interfaces. The SOA front-end components 1206A-B may also be configured to support multiple protocols and sessions such as an HTTP session, an HTTPS session, an SSL protocol session, a TLS protocol session, a DTLS protocol session, an FTP session, a UDP, a TCP, and an RDMA transfer protocol.

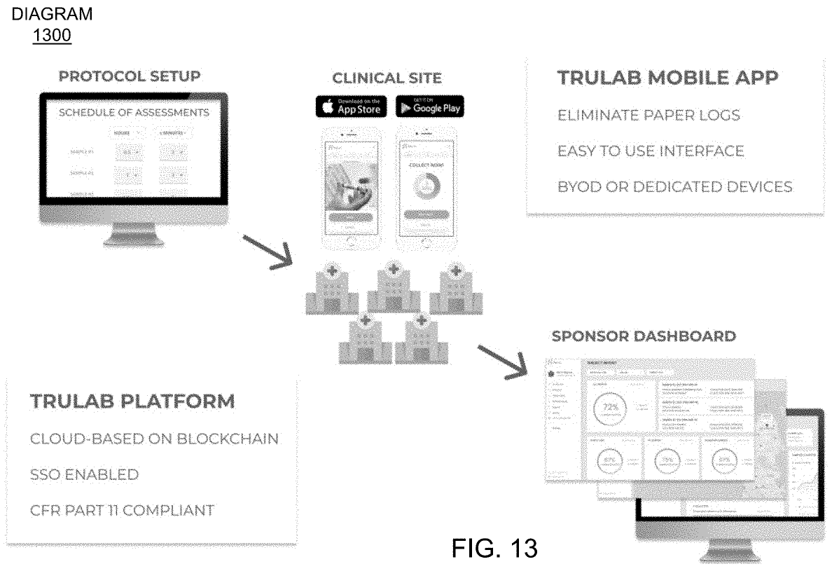

In summary, the TruLab system 600, (including the TruLab Server application 602, TruLab mobile app 610, and TruLab web based app 614) transforms computers into machines that solve the problem of management and tracking of collection, processing, and movement of biological samples during clinical trials as depicted in diagram 1300 of FIG. 13. The disclosed systems provide for protocol setup of clinical trials in including schedule of assessments and sponsor dashboards for monitoring of the clinical trials. The TruLab mobile app 610 eliminates paper requisition forms and paper logs with a user's personal smartphone or tablet via an easy to use interface. Overall the TruLab system (i.e. platform) provides cloud-based blockchain using single sign on (SSO). Additionally, the TruLab system is 21 Code of Federal Regulations (CFR) Part 11 compliant.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including object oriented and/or procedural programming languages. Programming languages may include, but are not limited to: Ruby, JavaScript, Java, Python, Ruby, PHP, C, C++, C#, Objective-C, Go, Scala, Swift, Kotlin, OCaml, AngularJS, or the like. The program code may execute entirely on the user's computer, partly on the user's computer, as a stand-alone software package, partly on the user's computer, and partly on a remote computer or entirely on the remote computer or server.

Aspects of the present invention are described in the instant specification with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. Thus, for example, reference to “a user” can include a plurality of such users, and so forth. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method implemented on one or more servers for providing management and tracking of collection, processing, and movement of a plurality of biological samples during clinical trials, the method comprising:
receiving first biological sample data from a first mobile device, wherein the first mobile device comprises:
a camera for capturing at least a portion of the first biological sample data; and
a first graphical user interface (GUI) configured for displaying the first biological sample data; and
storing the first biological sample data within a distributed ledger.

2. The method of claim 1, wherein the distributed ledger comprises at least one block chain.

3. The method of claim 1 further comprising:
retrieving the first biological sample data from the distributed ledger; and
transmitting the first biological sample data to a GUI on a second mobile device.

4. The method of claim 3 further comprising:
receiving second biological sample data from the second mobile device; and
storing the second biological sample data within the distributed ledger.

5. The method of claim 4 further comprising:
retrieving the first biological sample data and the second biological sample data from the distributed ledger; and
transmitting the first biological sample data and the second biological sample data to a third GUI on a third mobile device.

6. The method of claim 5, wherein the third GUI comprises a dashboard and the dashboard includes:
a visualization tool for graphically displaying the first biological sample data and the second biological sample data, and
a filtering tool for filtering the first biological sample data and the second biological sample data.

7. The method of claim 1, wherein the first biological sample data includes a first unique sample identifier associated with a first biological sample collected from a first clinical trial participant.

8. The method of claim 7, wherein the first unique sample identifier is at least one of a barcode and a quick response (QR) code.

9. The method of claim 7, wherein the first biological sample data further includes a first clinical trial subject identifier associated with the first clinical trial participant.

10. The method of claim 9, wherein the first biological sample data further includes a timestamp associated with a first collection time of the first biological sample.

11. The method of claim 10, wherein the first biological sample data further includes a first location identifier associated with a first capture location of the first biological sample.

12. The method of claim 11, wherein the first capture location is at least one of a clinical site, a central laboratory, a repository, and a bioanalytical laboratory.

13. The method of claim 1, wherein at least one server of the one or more servers is a virtual server.

14. The method of claim 13, wherein the virtual server hosted in a cloud computing environment.

15. The method of claim 1, wherein the first biological sample data is received via a first secure web portal provided for the first mobile device.

16. The method of claim 1, wherein the first GUI is provided by a first application specific program executing instructions on the first mobile device.

17. The method of claim 16, wherein the first mobile device is at least one of a smart phone and a tablet.

18. The method of claim 17, wherein the first application specific program is at least one of an iOS® app and an Android® OS app.

19. A server for providing management and tracking of collection, processing, and movement of biological samples during clinical trials, the server comprising
a memory;
a database; and
a processor configured for:
receiving first biological sample data from a first mobile device, wherein the first mobile device comprises:
a camera for capturing the first biological sample data; and
a first graphical user interface (GUI) configured for displaying the first biological sample data; and
storing the first biological sample data within a distributed ledger.

20. A non-transitory computer readable medium comprising a plurality of machine-readable instructions which when executed by one or more processors of a server are adapted to cause the server to perform a method server for providing for providing management and tracking of collection, processing, and movement of biological samples during clinical trials, the method comprising:
receiving first biological sample data from a first mobile device, wherein the first mobile device comprises:
a camera for capturing the first biological sample data; and
a first graphical user interface (GUI) configured for displaying the first biological sample data;
and
storing the first biological sample data within a distributed ledger.
